(19) United States
(12) Patent Application Publication
(10) Pub. No.: US 2013/0345516 A1
(43) Pub. Date: Dec. 26, 2013

(54) Shank for a flexible endoscope or a flexible endoscopic instrument

(71) Applicant: Gereon Kindler, Schaffhausen (CH)

(72) Inventor: Gereon Kindler, Schaffhausen (CH)

(21) Appl. No.: 13/916,213

(22) Filed: Jun. 12, 2013

(30) Foreign Application Priority Data
Jun. 12, 2012 (DE) 10 2012 105 061.9

(51) Int. Cl. A61B 1/005; A61B 17/00

(52) U.S. Cl. CPC A61B 1/0057; A61B 17/00234; A61B 1/0052
USPC 600/145; 600/149; 606/130; 600/147

(57) ABSTRACT

A shank for a flexible endoscope or a flexible endoscopic instrument includes a first shank portion and, arranged in the distal direction from the first shank portion, a second shank portion, wherein the second shank portion can be angled relative to a distal end area of the first shank portion by a longitudinal adjustment of at least one tensioning means extending inside the second shank portion, and wherein at least one cord drive is provided for the longitudinal adjustment of the tensioning means. In this way, a shank for a flexible endoscope or a flexible endoscopic instrument is created which is of simple and robust construction and which can be produced inexpensively. The invention also relates to a flexible endoscope and a flexible endoscopic instrument.

### FIELD OF THE INVENTION

The present invention relates to a shank for a flexible endoscope or a flexible endoscopic instrument and to a flexible endoscope and a flexible endoscopic instrument having such a shank.

### BACKGROUND OF THE INVENTION

Flexible endoscopes are used for many applications in medicine and technology. Flexible endoscopes of this kind comprise a flexible elongated shank, which is suitable for insertion into a cavity, for example a cavity inside a body or a cavity of a technical object. Generally, an endoscope lens is arranged at the tip of the endoscope shank to generate an image of a scene in the observed cavity. To record and send the endoscopic image from the distal end area (i.e. remote from the observer) to the proximal end area (i.e. near the observer) of the endoscope, it is possible, for example, to provide an organized bundle of light-guiding fibers extending inside the shank, or an electronic image recorder, for example a CCD chip, which is arranged in the area of the distal end of the shank, and of which the signals are transmitted to the proximal end area via electrical lines extending inside the shank. Since there is generally insufficient light in the observed cavity, a light-guiding system can also be arranged inside the shank in order to convey light to the distal end of the endoscope, where it is used to illuminate the cavity. Furthermore, the endoscope shank can have one or more working channels through which endoscopic working instruments are passed from the proximal to the distal end area of the shank, and with the aid of which manipulations can be performed inside the cavity.

Endoscopic instruments are also known to comprise a flexible elongated shank which is likewise suitable for insertion into a cavity, for example a cavity inside a body or a cavity of a technical object. A flexible endoscopic instrument of this kind can be used to perform manipulations in the cavity and, for this purpose, can be designed for example as a grasping instrument for gripping and manipulating tissue or articles in the cavity inside the body or in the cavity of the technical object. For this purpose, a tool is arranged at the distal end of the flexible shank and can be operated from the direction of the proximal end of the shank by way of a transmission means extending inside the shank. A flexible endoscopic instrument of this kind generally does not have a dedicated lens system for recording an endoscopic image, but it can in particular be used together with a flexible endoscope.

It is often desirable to be able to angle the distal end of the shank, i.e. the tip of the endoscope or of the endoscopic instrument, in order to make insertion of the endoscope or of the endoscopic instrument through a non-rectilinear channel easier, in order to be able to move the tip in a lateral direction inside a cavity, and in order to be able to vary the viewing direction of a lens system arranged in the endoscope tip or the working direction of a tool arranged at the tip of the endoscopic instrument. For this purpose, the shank has a controllable portion, in particular a controllable end portion, that can be actively angled by a desired amount in a desired direction and, for this purpose, can be controlled from the direction of the proximal end of the endoscope or the endoscopic instrument. The shank for a flexible endoscopic instrument does not itself need to have a tool and a transmission means, but instead can comprise, for example, a working channel into which a flexible endoscopic working instrument, which cannot be actively angled and which has such a tool, can be inserted as far as the distal end of the shank and optionally beyond this, such that the flexible working instrument can be angled with the aid of the shank.

To permit controllable angling of a portion of a shank of a flexible endoscope, it is known to design this with a base structure composed of individual, mutually pivotable segments that can be actuated by cable pulls or Bowden cables guided in the endoscope shank. For actuation, handwheels in particular are provided that are arranged on the handle of the endoscope. According to US 2005/0131279 A1, each segment of a controllable portion of the endoscope shank is mounted so as to be pivotable relative to the next and/or preceding segment. To angle the shank portion in question or the endoscope tip, four cable pulls are provided which are guided as far as the endoscope tip from a control mechanism arranged at the proximal end. The cable pulls extend in the edge area of the segments and are offset by 90° to each other relative to a longitudinal axis, such that control of the endoscope tip to a desired direction can be effected by rolling up the corresponding cables in the control mechanism.

However, the cables of such cable pulls have a tendency to lengthen after repeated use, which adversely affects the controllability of the endoscope tip or makes it necessary to regularly re-tension the cable pulls. Resetting devices for adjusting the tension of the cable pulls are known, for example from DE 29 14 748 C2 and U.S. Pat. No. 4,203,430. However, resetting devices of this kind are associated with increased outlay in terms of construction and control. The necessary permanent tensioning of the cable pulls can also lead to a shortening of the shank, which can have a deleterious effect on the controllability of the endoscope tip and indeed the properties of the endoscope shank, for example the flexibility or surface quality. The attainable angling also depends on the curvature of the whole shank portion through which the cable pulls are guided, with the result that there is no clear relationship between the position of a handwheel, with which a cable pull can be rolled up, and the attained deflection angle of the endoscope tip. Curvatures of the endoscope shank also cause increased friction of the cable pulls, which likewise makes it difficult to control the angle. Finally, the cable pulls, particularly in longer shanks, may sever on account of the friction and of corresponding wear.

JP 07134253 A discloses an endoscope with a curvable portion comprising a plurality of segments which are interconnected in a hinged manner and which have threaded nuts, the latter cooperating with bolts that are connected to a flexible shaft. Near a proximal end of the curvable portion, a micromotor is arranged in the shank of the endoscope and sets the flexible shaft in rotation in order to control angling of the curvable portion. This entails quite a considerable outlay in terms of construction. Moreover, the load-bearing capacity of the shaft with the associated threaded nuts, and in particular the load-bearing capacity of the thread, is not always sufficient in the case of small shank diameters. In addition, arranging a micromotor in an endoscope shank of small diameter, within which there extend further lines and channels, is not entirely easy, and it is also associated with considerable effort for reasons of accessibility to the motor for repair and maintenance purposes and, if appropriate, for cleaning and sterilization.

### SUMMARY OF THE INVENTION

The object of the present invention is to make available a shank for a flexible endoscope or a flexible endoscopic instrument, and also a flexible endoscope and a flexible endoscopic instrument, wherein the abovementioned disadvantages are avoided, and wherein the shank in particular is of simple construction, durable and robust and can be designed with a small diameter.

This object is achieved by a shank, a flexible endoscope, and/or a flexible endoscopic instrument according to the claims. Advantageous developments of the invention are also set forth in the claims.

A shank according to the invention for a flexible endoscope or for a flexible endoscopic instrument is elongated and is designed for insertion into a cavity, for example a cavity inside a body or a cavity of a technical object. The flexible endoscope can comprise a handle, which is arranged at a proximal end of the shank and can have control elements and/or connections for further appliances, for example a suctioning and flushing device, a light source or a camera unit. The handle can also contain a camera. The flexible endoscopic instrument can have a handle arranged at a proximal end of the shank with an actuating element for a tool arranged at the distal end of the shank. The flexible endoscope or the flexible endoscopic instrument is designed for use in medical procedures, particularly surgical procedures, or for technical applications.

The shank comprises a first shank portion and, arranged in the distal direction from the first shank portion, a second shank portion, which can adjoin a distal end area of the first shank portion. The shank can comprise further shank portions. The second shank portion can be designed, for example, as an endoscope tip that can be adjusted in terms of its angle. The second shank portion can be angled relative to the distal end area of the first shank portion. In particular, the second shank portion is designed to be actively curvable, while the first shank portion is at least partially flexible but not actively curvable.

Inside the second shank portion, at least one tensioning means is arranged that acts in the longitudinal direction of the second shank portion. Here, the term “tensioning means” comprises, for example, a wire or a cable pull that exerts a tensile force, but it can also be designed as a pushing means, for example as a stiff wire or as a wire guided in a sheath, for example as a Bowden cable, which is suitable for transmitting both pulling and pushing forces in the longitudinal direction of the second shank portion. The second shank portion can be angled relative to a distal end area of the first shank portion by a longitudinal adjustment of the tensioning means. The tensioning means interacts with a support structure of the second shank portion in such a way that a change in length or a lengthwise movement of the tensioning means causes an angling of the second shank portion, in particular a curving of the second shank portion. For this purpose, the tensioning means can be guided from a proximal end area to a distal end area of the second shank portion and can be connected to the support structure at least in the distal end area. The support structure can consist of a sequence of segments connected to one another in a hinged manner, wherein the tensioning means, laterally offset with respect to the hinges, engages on at least one end segment of the support structure.

According to the invention, at least one cord drive is arranged for the longitudinal adjustment of the at least one tensioning means. For this purpose, the cord drive can be a part of the tensioning means or can engage thereon. A cord drive, which is also referred to as a twisting drive, comprises a plurality of cords or wires which are arranged twisted together, or a single cable braided from a plurality of cords and twistable about itself. The cords or wires to be twisted together can also themselves be braided from a plurality of cords or wires, as a result of which increased tensile strength can be achieved. Where reference is made below to “twisting cords”, this term also signifies twisting wires or a cable braided from a plurality of cords. Depending on the degree of twisting or the number of rotations for twisting, the effective length of the cord drive varies. The change in length is also dependent on the diameter and the number of cords or wires that are to be twisted together. The cord drive can be driven, for example, by a shaft which can extend in the longitudinal direction of the second or end area of the first shank portion, or also by a motor arranged inside the shank.

By virtue of the fact that a cord drive is provided for the longitudinal adjustment of the tensioning means, the second shank portion of the shank can be angled with sufficient force even in the case of a small shank diameter. Moreover, the cord drive is of simple and robust construction and can be produced inexpensively.

To control the longitudinal adjustment of the tensioning means and therefore the angling of the second shank portion, actuating means are preferably provided which are arranged in the proximal direction from the first shank portion and, for example, can be in the form of a handwheel arranged on a handle or in the form of a motor arranged in a handle. Moreover, to control the angle of the second shank portion relative to the distal end area of the first shank portion, at least one torsion shaft is arranged inside the first shank portion, by means of which an actuating movement generated by the actuating means can be transmitted to the second shank portion or to an end area of the first shank portion, in order to drive the at least one cord drive and thereby effect a longitudinal adjustment of the at least one tensioning means. The torsion shaft is elongated, continuous from the proximal end area to the distal end area of the first shank portion, and flexible at least in parts. The torsion shaft can be guided inside the first shank portion in an edge area of the cross section, such that a sufficient cross-sectional area remains for the passage of further channels or electrical lines and light lines. By means of the rotation of the torsion shaft coupled to the cord drive, it is thus possible to control the longitudinal adjustment of the tensioning means for controlling the angle of the second shank portion.

By virtue of the fact that the longitudinal adjustment of the tensioning means can be controlled by actuating means that are arranged at the proximal end and that generate a rotation movement which is transmitted to the cord drive via at least one torsion shaft extending inside the first shank portion, an actuating movement for controlling the angle of the second shank portion can be generated by easily controllable actuating means, which actuating movement can easily be transmitted through a shank designed with a small diameter. The actuating movement is transmitted to the second shank portion more directly than would be possible with cable pulls or Bowden cables. No tensile force or pressure force is exerted on the first shank portion, which generally makes up most of the overall length of the shank, such that compression of the shank is avoided. Moreover, re-tensioning of cable pulls, which could lengthen over the course of time, is unnecessary. Furthermore, the actuating movement transmitted as far as the second shank portion is substantially independent of the length and curvature of the first shank portion, such that the controllability of the angle of the second shank portion is improved. In this way, a simply constructed flexible shank for a flexible endoscope or for a flexible endoscopic instrument is created, in which a distal shank portion which can be angled can be controlled independently of a curvature of a proximal shank portion with actuating means arranged at the proximal end, and which can also be realized with a shank diameter of, for example, less than 6 mm, preferably 4 mm or less.

According to a preferred embodiment of the invention, the torsion shaft is connected to the tensioning means, for the purpose of controlling the longitudinal adjustment of the tensioning means, via a cord drive. Such a cord drive comprises two twisting cords which are twisted around each other in a starting position and extend substantially in the longitudinal direction and which are each held in a proximal and a distal connector piece. The proximal connector piece is connected to the distal end of the torsion shaft in a rotationally fixed manner and is rotatable therewith, but is mounted in the endoscope shank so as to be supported in the longitudinal direction, whereas the distal connector piece is rotationally fixed but longitudinally movably guided inside the endoscope shank and is connected to a proximal end of the tensioning means guided inside the second shank portion. A rotation of the torsion shaft in a first direction leads to a further twisting of the twisting cords about one another and thereby to a shortening, as a result of which the distal connector piece moves toward the proximal connector piece and thus exerts a tension on the tensioning means in the proximal direction in its proximal area. If the starting position of the twisting cords corresponds to a rectilinear, i.e. unangled state of the second shank portion, this causes an angling of the second shank portion. A rotation of the torsion shaft in the opposite direction leads to an untwisting of the twisting cords, such that a reduction of the angling can be effected by an elastic restoring force or by a counteracting tensile force applied to an opposite side of the shank. The cord drive can be arranged in a proximal end area of the second shank portion, in a distal end area of the first shank portion, in both shank portions, or in a transition portion between the first and second shank portions. The transition portion does not necessarily have to be flexible. By virtue of the fact that a longitudinal movement and thus a longitudinal adjustment of the tensioning means is effected via a cord drive, a conversion of the rotation movement of the torsion shaft into a longitudinal adjustment of the tensioning means is possible in a particular simple way for controlling the angle of the second shank portion.

According to another preferred embodiment of the invention, the tensioning means is itself designed, at least in parts, as the cord drive or as twisting cords of the cord drive. According to this embodiment, the torsion shaft is connected to a proximal connector piece for rotation therewith, in which at least two twisting cords are secured which are guided through the second shank portion to the distal end area thereof and are held there in a distal connector piece connected to a support structure of the second shank portion, for example to an end segment. The proximal connector piece is mounted rotatably on the torsion shaft and can be driven by the latter, while the distal connector piece is connected in a rotationally fixed manner to the shank, in particular to the second shank portion. Both connector pieces are supported against a longitudinal movement in the respective area of the endoscope shank. By means of a rotation of the torsion shaft, the proximal connector piece thus rotates relative to the distal connector piece, as a result of which the twisting cords are twisted relative to each other and the length of the tensioning means, formed at least in part by the twisting cords, is changed. Proceeding from a starting position in which the twisting cords are twisted together to a certain extent, further twisting causes shortening of the tensioning means, on account of a corresponding rotation of the torsion shaft, whereas a rotation in the opposite direction permits lengthening. In this way, shortening or lengthening, and therefore a length adjustment, of the tensioning means is effected, wherein a longitudinal guide of one of the connector pieces and a separate tensioning means are not necessary. In this way, control of the angling of the second shank portion is made possible in a particularly simple manner by a rotation of the torsion shaft.

The second shank portion preferably has a plurality of tensioning means which are arranged lying opposite each other in pairs. During angling of the second shank portion, two tensioning means thus act counter to each other, wherein the respective longitudinal adjustments of the tensioning means act counter to each other. In this way, it is possible to achieve angling in different directions with a high degree of precision and with an always sufficient force, since in each direction of angling one of the tensioning means is shortened or the distal end of one of the tensioning means is pulled in the proximal direction.

In one embodiment, two pairs of tensioning means can be provided which are offset by 90° to each other relative to a longitudinal axis of the second shank portion, in order to allow the second shank portion to be angled in mutually perpendicular directions. It is thus possible, in a particularly simple way, to obtain angling to the left or to the right, as seen by an observer, also upward or downward, and, by a simultaneous actuation of the pairs of tensioning means, angling in any direction lying therebetween.

In another embodiment, it is possible to provide three tensioning means offset in each case by about 120° relative to the longitudinal axis. In this way too, angling can be achieved in all directions.

A plurality of torsion shafts are advantageously provided which interact with the plurality of tensioning means to control an angling of the second shank portion. In particular, each torsion shaft can be connected to a respective tensioning means or a respective cord drive. It is thus possible, in a simple way, to angle the second shank portion in different directions with a high degree of precision.

According to a preferred embodiment of the invention, the plurality of torsion shafts are interconnected via a toothed gearing, in such a way that tensioning means lying opposite one another are each movable in opposite directions. In particular, the toothed gearing can be arranged at a proximal end of the torsion shaft, wherein torsion shafts assigned to two cord drives lying opposite each other are coupled to each other. By means of the toothed gearing, a coupling is possible of the kind by which a lengthening of one tensioning means leads to a simultaneous shortening of a tensioning means lying opposite and acting counter to the former tensioning means, or a movement of one tensioning means in the proximal direction has the effect that a tensioning means lying opposite and acting counter to the former tensioning means is moved in the distal direction. For this purpose, for example, each of the two torsion shafts acting counter to each other can carry a rotationally fixed toothed wheel at its proximal end, wherein the two toothed wheels mesh with each other, such that the torsion shafts always rotate in opposite directions and cause opposite changes in length of cord drives twisted in the same direction. If the two corresponding cord drives are twisted in different directions in the starting position, an uneven number of toothed wheels can be interposed between the two toothed wheels connected to the torsion shafts, such that the two torsion shafts rotate in the same direction and cause opposite changes in length of the cord drives twisted in the same direction. In this way, the controllability of the angling of the second shank portion is further improved.

Since a cord drive generally has a non-linear relationship between twisting and change in length, it is particularly advantageous if the starting position of both cord drives acting counter to each other, which position can correspond to a rectilinear state of the second shank portion, is chosen in a twisting range in which a linear relationship exists at least in part between the angle or number of revolutions of a further twisting or untwisting on the one hand and the resulting change in length on the other hand. In this way, the angling of the second shank portion can be more precisely controlled and, in particular, a desired angle can be more precisely achieved. Moreover, in a particularly simple way, at least within a limited angle range, it is thus possible to achieve a low-tension counteraction of the cord drives lying opposite each other and of the torsion shafts coupled thereto for angling the second shank portion in different directions. To permit a greater range of angling, the tensioning means or the cord drives can be designed to be elastically extensible and are pre-stressed.

The cord drive preferably comprises at least two twisting cords made from a polymer material. Such a polymer material can consist of polyethylene (PE) fibers, as are obtainable, for example, under the name “Dyneema®” from the company called DSM. Such a cord has a sufficient load-bearing capacity and is permanently flexible and thus continuously permits exact control of the angling of the second shank. A twisting cord braided from a plurality of cords can, for example, have a diameter of approximately 0.15 mm; two twisting cords can thus be guided, as in a Bowden cable, in a sheath with an internal diameter of approximately 0.30 mm.

An endoscope according to the invention comprises an above-described shank, which is designed as an endoscope shank. Light guides, image guides, suctioning and flushing channels and/or one or more working channels for endoscopic working instruments can extend through the shank.

An endoscopic instrument according to the invention comprises an elongated shank, which is designed as described above. Moreover, a tool can be arranged at the distal end of the shank of the endoscopic instrument, wherein the tool can be controlled from the direction of the proximal end of the shank, for example with the aid of grips arranged at the proximal end, via a transmission means, for example a tensioning means, extending inside the shank. The shank of the endoscopic instrument can, for example, comprise suctioning and flushing channels, and also a working channel through which a non-actively controllable endoscopic working instrument can be inserted.

Preferably, the flexible endoscope or the flexible endoscopic instrument is designed in such a way that the at least one torsion shaft can be actuated via at least one control element arranged in a proximal area of the endoscope or of the endoscopic instrument, in particular on a handle, and designed as a handwheel, lever or crank, for example, and/or via a motor, which can be arranged in the handle, for example. By means of the control element and also by means of the motor, the torsion shaft can be set in rotation in a manner sufficient for a precise control of the angling. However, the motor can also be arranged in a motor unit separate from the endoscope or the endoscopic instrument and, if appropriate, from the handle, in which case the rotation movement can be transmitted to the endoscope shank via a flexible shaft. The actuation via a motor, which can be controlled by a control device, is also particularly advantageous if the tensioning means are not arranged lying opposite each other in pairs, but are instead arranged offset by 120° to each other, for example.

According to another preferred embodiment of the invention, the flexible endoscope or the flexible endoscopic instrument comprises a handle, wherein the shank can be connected releasably to the handle via a coupling. An endoscopic system can therefore comprise a plurality of shanks of different sizes, in particular of different diameters and lengths, and if appropriate of different construction, which can be connected to one handle which, for example, can comprise the drive for the angling and a camera. By virtue of the fact that, when coupling the shank to the handle, no cable pulls extending in the longitudinal direction of the shank have to be connected to the handle, it is not necessary for these to be readjusted or re-tensioned after coupling. By contrast, the transmission of the actuating movement of the cord drive via a torsion shaft permits a simpler connection, which merely has to be designed fixed in rotation and can be easily separated in the longitudinal direction. The drive and a control device are arranged in the handle. In this way, an endoscope or an endoscopic instrument and/or an endoscopic system is created which can be used in a versatile way but which, on account of the handle that can be used with a plurality of shanks, is relatively inexpensive and requires little effort for changing the shanks.

In one embodiment, the handle comprises a motorized drive of the at least one torsion shaft or of the cord drive. When the shank is uncoupled from the handle, a rotary position of the at least one torsion shaft is memorized, and a torsion-shaft coupling of the handle can be controlled for coupling the shank in such a way that, when coupled, it assumes a rotary position corresponding to the memorized rotation position of the torsion shaft. This permits simple coupling of the shank to the handle. Moreover, the memorizing of the rotation position of the torsion shaft permits a precise control of the angling of the second shank portion even in the case when a non-linear relationship exists between the rotation position of the torsion shaft and the angling. In this case, the non-linear relationship can be memorized in a control device and, on the basis of the rotation position of the torsion shaft, a respectively current rotation position of a cord drive can be determined, and the angling can thus be precisely controlled taking into account the non-linear relationship. In this way, the respectively current angling of the second shank portion can also be determined by the control device, such that, for example in a virtual view of the endoscope or the endoscopic instrument and of the observed cavity, the spatial position of the endoscope tip can be correctly viewed.

The rotary position of the at least one torsion shaft, which position is adopted by the latter upon separation from the corresponding motor shaft, can advantageously be memorized in a memory assigned to the shank. In this way it is possible that, when connecting the shank to a handle, the coupling of the motor shaft can be rotated to the correct position, and the control device has the necessary information for precise control of the angling, even in the case where the shank is connected to another handle.

To determine the rotary position of the torsion shaft upon separation from the handle, a sensor can be provided, which is arranged in a proximal end area of the shank. The sensor may comprise a potentiometer or another rotary encoder, such as an absolute value sensor or an optical multi-turn rotary encoder. The revolutions and the covered angle of the torsion shaft are measured by the sensor. Upon separation from the drive, the sensor can remain in the corresponding rotation position, such that a read-out is possible at any time. In this way, the revolutions executed during the operation of the endoscope or of the endoscopic instrument for angling the second shank portion can be counted, and the rotary position of the torsion shaft can be determined.

During the use of the endoscope according to the invention or the endoscopic instrument according to the invention, for the purpose of controlling a curvable shank portion, a curvable tip of the endoscope, or the working direction of a tool arranged at the tip of the endoscopic instrument with the aid of a control element or motor, a torsion shaft extending in a first shank portion arranged at the proximal end is thus rotated and, in this way, a tensioning means extending in a second portion of the shank is adjusted in its longitudinal direction, i.e. shortened, lengthened, or moved. Through the longitudinal adjustment of the tensioning means, the second shank portion is curved and, in this way, the endoscope tip or the tip of the endoscopic instrument is angled.

If another endoscope shank is to be used with the same handle of the flexible endoscope or the same shank is to be used with another handle, the shank connected to the handle is separated therefrom. Since the torsion shaft only has to be connected for rotation to a motor shaft assigned to the handle, assembly is achieved in a simple way. The same applies to the shank and the handle of a flexible endoscopic instrument. The rotation of the torsion shaft can be determined by a sensor and memorized in a memory, arranged in the proximal end area of the shank, upon separation of the shank from the handle. When connecting another shank to the handle or connecting the shank to another handle, the rotary position memorized in the memory of the shank to be connected is transmitted electrically, mechanically or by wireless transmission means, and the motor shaft is brought to a suitable position such that the connection can take place in a simple way. Moreover, the transmission of the rotary position permits a calculation of the current setting and a precise control of the endoscope tip or the tip of the endoscopic instrument.

It will be appreciated that the aforementioned features and the features still to be explained below can be used not only in the indicated combinations but also in other combinations or singly, without departing from the scope of the present invention.

### BRIEF DESCRIPTION OF THE DRAWINGS

Further aspects of the invention will become clear from the following description of a preferred illustrative embodiment and from the attached drawing, in which:

FIG. 1a and FIG. 1b each show an overall view of a flexible endoscope according to the invention in an illustrative embodiment;

FIG. 2 shows a schematic longitudinal section through an end area of a shank according to a first illustrative embodiment of the invention, with only one half of the longitudinal section being depicted;

FIG. 3 shows a schematic longitudinal section through an end area of a shank according to a second illustrative embodiment of the invention, with only one half of the longitudinal section being depicted;

FIG. 4 shows a schematic view of a coupling mechanism between an endoscope shank and a handle according to an illustrative embodiment of the invention.

### DETAILED DESCRIPTION OF THE INVENTION

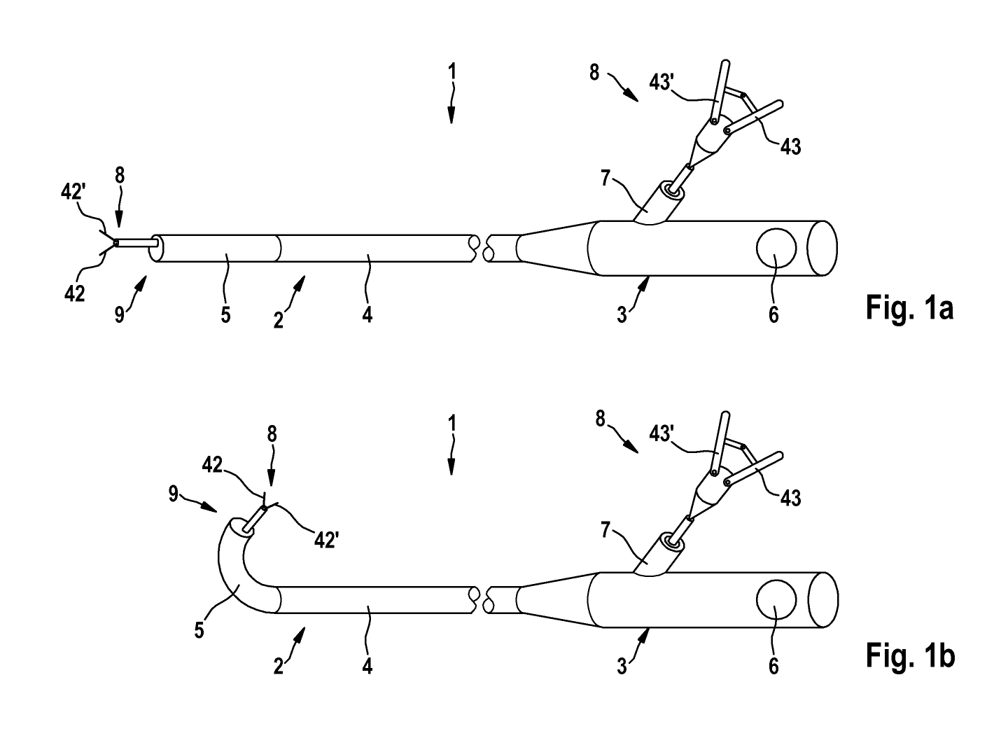

As is shown in FIG. 1a and FIG. 1b, a flexible endoscope 1 according to one embodiment of the invention comprises a flexible endoscope shank 2 and a handle 3. The endoscope shank 2 comprises a first shank portion 4 and a second shank portion 5. The second shank portion 5 is the distal end portion of the endoscope shank 2. The first shank portion 4 is the proximal part of the endoscope shank 2, which in the example shown occupies the entire remaining length of the endoscope shank 2 and is considerably longer than the second shank portion 5. A control element 6, indicated symbolically, is arranged on the handle 3 and is used to control angling of the second shank portion 5. The control element 6 can be designed as a handwheel, lever or crank, or can also be a control element, for example for controlling a motor. Moreover, the handle 3 has a guide tube 7 into which a controllable endoscopic working instrument 8, shown by way of example but not part of the invention, can be inserted and guided through a working channel, which extends within the endoscope shank 2 to the distal end 9 of the endoscope shank 2, and out of and beyond the working channel. At its distal end, the working instrument 8 has a tool which is designed, for example, for gripping or cutting and which has two jaw parts 42, 42′ that act against each other and are actuated via grips 43, 43′ arranged at the proximal end.

FIG. 1a shows the endoscope shank 2 in a rectilinear state, while FIG. 1b shows the endoscope shank 2 in an angled state. The instrument 8 and the first shank portion 4 may be flexible but not actively controllable. By contrast, the second shank portion 5 of the endoscope shank 2 is actively curvable, and thus controllable, by actuation of the control element 6. In its interior, the second shank portion 5 has a support structure composed of shank segments which are connected to one another in a hinged manner and which, to permit the angling, interact with at least two mutually opposite tensioning means guided inside the second shank portion 5. Each of the tensioning means is connected to the handle 3 and to the control element 6 via a respective torsion shaft, which extends through the first shank portion 4. The endoscope shank 2 can be separated from and reconnected to the handle 3 via a coupling (not shown).

In some areas, the endoscope shank 2 can be strengthened by a surrounding metal braid. The first shank portion 4 and the second shank portion 5 can be surrounded by a flexible sheath which, for example, can be a tubing made of a plastics material. The distal end area of the second shank portion 5 can be rigid and can receive optical and electronic components, for example an endoscope lens and, if appropriate, an electronic imaging device (not shown).

A flexible endoscopic instrument according to one embodiment of the invention is constructed as the flexible endoscope 1 shown in FIG. 1a and FIG. 1b, but without a lens system. The flexible endoscopic instrument can comprise the endoscopic working instrument 8 or can also be designed as an endoscopic guide instrument for guiding and controlling a non-controllable endoscopic working instrument 8.

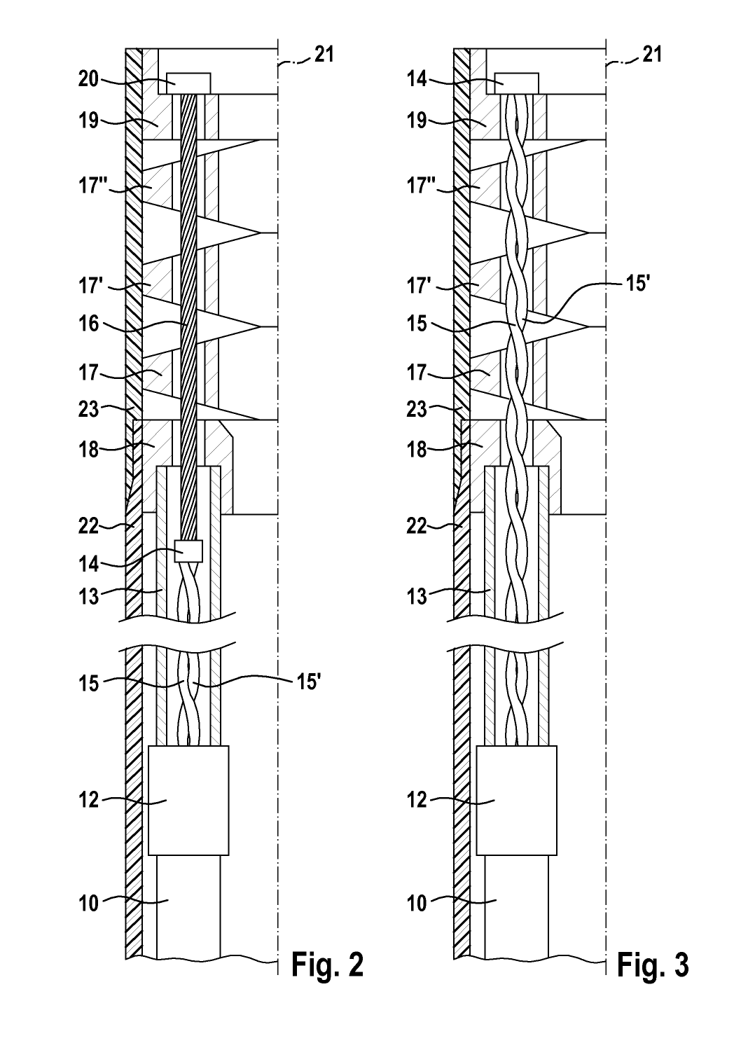

FIG. 2 shows, in longitudinal section, an embodiment of the invention with a tensioning means designed as a cable pull for the second shank portion 5 of the endoscope shank 2 (see FIG. 1), wherein the tensioning means is connected by a cord drive to the torsion shaft 10 arranged in the first shank portion 4. The figure shows only the half of the longitudinal section situated on one side of the longitudinal axis. At its proximal end, the torsion shaft 10 can be driven in rotation by a motor, in particular by an electric motor arranged in a handle (not shown). The torsion shaft 10 is elongated, thin and rotationally stiff. The torsion shaft 10 can be made of Nitinol, for example. The torsion shaft 10 can be designed, for example, as a thin, flexible tube. The torsion shaft 10 has a distal end connected to a proximal connector piece 12 for rotation therewith, wherein the connector piece 12 is mounted rotatably inside the first shank portion 4. Against a longitudinal movement, particularly in the distal direction, the proximal connector piece 12 bears on a guide 13. A distal connector piece 14 is mounted so as to be longitudinally movable in the guide 13. The distal connector piece 14 can be secured against rotation by the guide 13 and/or by the tensioning wire 16 described below, which can be substantially rotationally stiff, and by the retainer 20 mounted for conjoint rotation in the end segment 19. The proximal connector piece 12 and the distal connector piece 14 are connected by two twisting wires 15, 15′ which extend substantially parallel to each other and which are each held in the proximal connector piece 12 and in the distal connector piece 14. To hold the twisting wires 15, 15′, it is possible, for example, for a micro-bolt to be provided in the proximal connector piece, around which micro-bolt a wire is wound whose two ends form the twisting wires 15, 15′, The cord drive, which is formed by the connector pieces 12, 14, the guide 13 and the twisting wires 15, 15′, is arranged in a distal end area of the first shank portion 4. Without appreciably limiting the functionality of the endoscope, the first shank portion 4 can be rigid or designed with a slight flexibility, thereby permitting a simple design of the cord drive. The guide 13 is preferably designed as a flexible tube.

The distal connector piece 14 is connected in a tension-resistant manner to a tensioning wire 16 which is guided in an edge area of the second shank portion. The second shank portion 5 has a support structure of which, in FIG. 2, three segments 17, 17′, 17″ are shown symbolically along with a proximal end segment 18 and a distal end segment 19. The guide 13 of the cord drive is fixed or mounted in the proximal end segment 18. The tensioning wire 16 is held in the distal end segment 19 by a retainer 20. The distal end segment 19 can have further components, for example an endoscope lens and/or outputs of channels extending inside the endoscope shank. In the edge area of the segments 18, 17, 17′, 17″, 19, the tensioning wire 16 is guided through corresponding openings or bores. As can be seen in FIG. 2, the retainer 20 of the tensioning wire 16 is offset radially relative to a longitudinal axis 21 of the second shank portion 5. Therefore, the retainer 20 is also offset radially relative to the pivot axes with which the segments 18, 17, 17′, 17″, 19 are mounted pivotably relative to one another and are substantially perpendicular to the longitudinal axis 21 and intersect the latter, and which are preferably perpendicular to each other in alternation, such that the differently directed pivot axes permit angling in ail directions.

If the torsion shaft 10 is now set in rotation by the motor (not shown in FIG. 2), the twisting wires 15, 15′ are entrained by the proximal connector piece 12 and are twisted relative to the distal connector piece 14, which is guided in conjoint rotation in the endoscope shank, and thus also relative to each other. During a rotation in a first direction of rotation, the twisting wires 15, 15′ are twisted further relative to the starting position shown in FIG. 2, which corresponds to a rectilinear position of the second shank portion 5. A shortening thus takes place, such that the distal connector piece 14 is moved in the proximal direction along the guide 13 toward the proximal connector piece 12, and the tensioning wire 16 is moved in the proximal direction relative to the proximal end segment 18. The twisting wires 15, 15′, for example at a length of approximately 60-80 mm, can have a tensioning path of approximately 8 mm, which is achieved after approximately 50 revolutions. For this purpose, an untwisted cord length of around 50 to 200 mm is needed, in particular more than 100 mm. On account of the extra-axial arrangement of the retainer 20, this causes a tilting of the distal end segment 19 and, depending on the design of the hinged connection between the segments 18, 17, 17′, 17″, 19, also a corresponding tilting of the segments 17, 17′, 17″. This results in a curving of the second shank portion 5 and therefore an angling of the second shank portion 5 or of the endoscope tip formed by the distal end segment 19.

A reverse rotation of the torsion shaft leads to a relaxation of the twisting wires 15, 15′. If a further similar mechanism as shown in FIG. 2 is arranged on the side of the endoscope shank lying radially opposite the side shown in FIG. 2, a twisting of the twisting wire on the opposite side can bring about an angling of the second shank portion 5 in the opposite direction, which the tensioning wire 18, the second connector piece 14 and the twisting wires 15, 15′ follow with corresponding untwisting, for example on account of the proximal coupling described below. If the retainer 20, the tensioning wire 16 and the guide of the tensioning wire 16 are correspondingly designed in the shank segments 18, 17, 17′, 17″, 19, a shearing force can also be exerted for angling the second shank portion 5 in the opposite direction. To permit angling of the second shank portion 5 in all directions, four cord drives acting opposite to each other in pairs can each be provided with a torsion shaft 10, these being offset from one another by 90° relative to the longitudinal axis 21. An outer sheath 22, which encloses the first shank portion, and the outer sheath 23 connected thereto and enclosing the second shank portion, are also indicated in FIG. 2.

FIG. 3 shows, again as half a longitudinal section, an embodiment of the invention in which no tensioning wire is needed to effect the angling of the second shank portion 5. Instead of the tensioning wire, the twisting wires 15, 15′ are guided in the second shank portion 5. As has been described for FIG. 2, a torsion shaft 10 extends inside the first shank portion 4 and can be driven at its proximal end by an electric motor. The torsion shaft 10 drives a proximal connector piece 12 in which the twisting wires 15, 15′ are secured. The proximal connector piece 12 is mounted rotatably in relation to a proximal end segment 18 of the second shank portion but is not longitudinally movable at least in the distal direction. At their distal end, the twisting wires 15, 15′ are held in a distal connector piece 14, which is secured for conjoint rotation on a distal end segment 19 and is supported against a longitudinal movement at least in the proximal direction. The twisting wires 15, 15′ are guided in corresponding openings or bores in the edge areas of the shank segments 17, 17′, 17″, of the distal end segment 19, and of the proximal end segment 18, in which the proximal connector piece 12 is mounted rotatably but in a tension-resistant manner in the distal direction. The twisting wires 15, 15′ or the cord drive can be guided in a flexible sheath; the proximal connector piece 12 is arranged rotatably relative to this sheath. As is shown in FIG. 3, the cord drive can be longer than the second shank portion 5, in order to maximize the angling that can be achieved.

By a further twisting of the twisting wires 15, 15′ on account of a rotation of the torsion shaft 10, going beyond the starting position shown in FIG. 3 and corresponding to a rectilinear state of the second shank portion, an axial tension is exerted on the distal connector piece 14 in the proximal direction. On account of the extra-axial arrangement of the distal connector piece 14, this brings about an angling of the second shank portion. A reverse rotation of the torsion shaft leads to a relaxation of the twisting wires 15, 15′, such that angling in the reverse direction can be brought about by, for example, a similarly constructed mechanism arranged on the radially opposite side. The embodiment shown in FIG. 3, in which the tensioning means itself is designed as a cord drive and the twisting wires 15, 15′ themselves serve as tensioning wire, thus works similarly to the embodiment shown in FIG. 2, but does not require a separate cable pull and is therefore a simple construction.

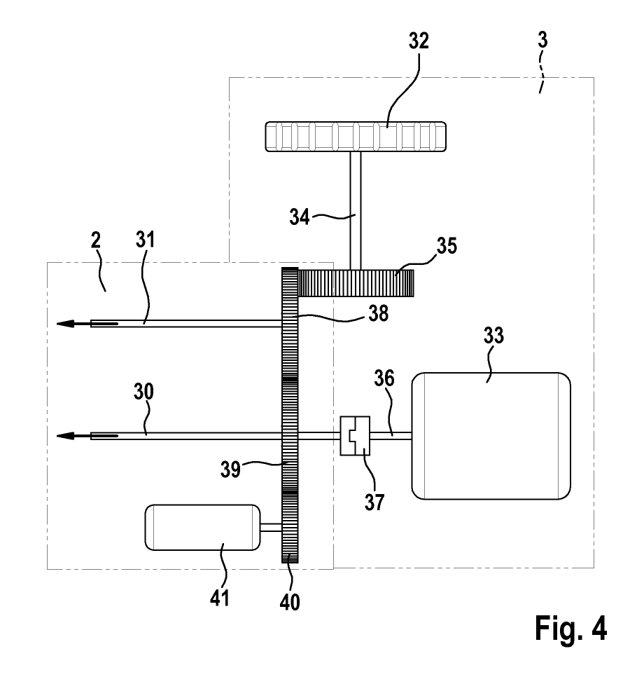

As shown symbolically in FIG. 4, an endoscope shank 2 can have a first torsion shaft 30 and a second torsion shaft 31, which can be driven by both a handwheel 32 and also a motor 33. The handwheel 32 is connected to a toothed wheel 35 by a shaft 34. A step-down gear (not shown in FIG. 4) can be provided, as a result of which, for example, one revolution of the handwheel causes 5 to 10 revolutions of the torsion shafts. The motor 33 drives a motor shaft 38, which has a coupling 37. The coupling can be designed, for example, as a slipping coupling, magnetic coupling, power take-off shaft, or in another way known per se. Handwheel 32, motor 33, shafts 34 and 36, toothed wheel 35, and coupling 37 may be disposed in a handle 3 that can be applied to the endoscope shank 2.

Upon assembly of the handle 3 to the endoscope shank 2, the toothed wheel 35 meshes with a toothed wheel 38, which is connected to the torsion shaft 31 for rotation therewith and drives the latter. The torsion shaft 30 is driven directly by the coupling 37. The toothed wheel 38 meshes with the toothed wheel 39, which sits on the torsion shaft 30 for rotation therewith and in turn drives the toothed wheel 40 connected to a rotary encoder 41. The relative rotation of the torsion shafts 30, 31 is defined by the connection of the toothed wheels 38, 39. The winding directions of the respective twisting wires are adapted to each other such that a shortening on one side of the second shank portion causes a corresponding lengthening of the drive acting on the other side of the second shank portion. The rotary encoder 41 registers the rotation of the toothed wheel 40, from which a rotation position of the torsion shafts 30, 31 can be determined. This rotation position is stored in a memory (not shown) and, when the shank 2 is coupled to the handle 3, is sent to a control device (likewise not shown) of the motor 33. In this way, it is possible at any time to achieve a control of the motor 33 corresponding to the actual setting of the drives.

For the sake of clarity, not all the reference numerals are shown in all of the figures. Reference numerals not explained in connection with one figure have the same meaning as in the other figures.

1. A shank for a flexible endoscope or for a flexible endoscopic instrument, the shank comprising:
a first shank portion,
a second shank portion arranged in a distal direction from the first shank portion,
at least one tensioning means extending inside the second shank portion, the at least one tensioning means being longitudinally adjustable to angle the second shank portion relative to a distal end area of the first shank portion, and
at least one cord drive providing longitudinal adjustment of the at least one tensioning means.

2. The shank according to claim 1, characterized in that the cord drive comprises at least one twisting cord.

3. The shank according to claim 2, characterized in that the cord drive comprises at least two twisting cords twisted around each other in a starting position.

4. The shank according to claim 1, further comprising at least one actuating means arranged in the proximal direction from the first shank portion, the at least one actuating means controlling the longitudinal adjustment of the at least one tensioning means via at least one torsion shaft, wherein the at east one torsion shaft extends inside the first shank portion.

5. The shank according to claim 4, characterized in that the at least one tensioning means is designed as a cable pull and is connected to a distal end of the torsion shaft via the cord drive.

6. The shank according to claim 5, characterized in that the at least one tensioning means comprises twisting wires or twisting cords of the at least one cord drive, which is connected to the distal end of the torsion shaft.

7. The shank according to claim 1, characterized in that the second shank portion has a plurality of tensioning means which are disposed opposite one another in pairs.

8. The shank according to claim 7, characterized in that the first shank portion has a plurality of torsion shafts which interact with the plurality of tensioning means in order to control angling of the second shank portion relative to the distal end area of the first shank portion in different directions.

9. The shank according to claim 8, characterized in that the plurality of torsion shafts are interconnected via at least one toothed gearing, in such a way that the tensioning means lying opposite one another are longitudinally adjustable in opposite directions.

10. The shank according to claim 1, characterized in that the cord drive has at least two twisting wires or twisting cords made from a polymer material.

11. A flexible endoscope having a shank according to claim 1.

12. A flexible endoscopic instrument having a shank according to claim 1.

13. The flexible endoscope according to claim 11, characterized in that the torsion shaft is actuated via at least one control element arranged in a proximal direction from the first shank portion and/or via a motor arranged in the proximal direction from the first shank portion.

14. The flexible endoscope according to claim 11, characterized in that the flexible endoscope comprises a handle arranged in a proximal direction from the first shank portion, and in that the shank is releasably connected to the handle.

15. The flexible endoscope according to claim 14, characterized in that the handle includes a motorized drive of the at least one torsion shaft, in that a rotary position of the at least one torsion shaft is memorized when the shank is separated from the handle, and in that a torsion-shaft coupling of the handle is controlled accordingly for coupling the shank.

16. The flexible endoscope according to claim 14, further comprising a memory for storing a rotary position of the at least one torsion shaft.

17. The flexible endoscope according to claim 16, further comprising a potentiometer or a rotary encoder for determining the rotary position.

18. The flexible endoscopic instrument according to claim 12, characterized in that the torsion shaft is actuated via at least one control element arranged in a proximal direction from the first shank portion and/or via a motor arranged in the proximal direction from the first shank portion.

19. The flexible endoscopic instrument according to claim 12, characterized in that the flexible endoscopic instrument comprises a handle arranged in a proximal direction from the first shank portion, and in that the shank is releasably connected to the handle.

20. The flexible endoscopic instrument according to claim 19, characterized in that the handle includes a motorized drive of the at least one torsion shaft, in that a rotary position of the at least one torsion shaft is memorized when the shank is separated from the handle, and in that a torsion-shaft coupling of the handle is controlled accordingly for coupling the shank.

21. The flexible endoscopic instrument according to claim 19, further comprising a memory for storing a rotary position of the at least one torsion shaft.

22. The flexible endoscopic instrument according to claim 21, further comprising a potentiometer or a rotary encoder for determining the rotary position.
